(19) United States
(12) Patent Application Publication
(10) Pub. No.: US 2022/0313166 A1
(43) Pub. Date: Oct. 6, 2022

(54) INTEGRATED PHYSIOLOGICAL MONITORING AND TISSUE ANCHORING DEVICE

(71) Applicant: Celero Systems, Inc., Lincoln, MA (US)

(72) Inventors: Benjamin David Pless, Lincoln, MA (US); Daniel Bacher, Walpole, MA (US)

(21) Appl. No.: 17/705,431

(22) Filed: Mar. 28, 2022

Related U.S. Application Data
(60) Provisional application No. 63/169,401, filed on Apr. 1, 2021.

(51) Int. Cl. A61B 5/00; A61B 17/08; A61B 5/1455; A61B 5/1459; A61M 5/172

(52) U.S. Cl. CPC A61B 5/6847; A61B 17/08; A61B 5/14552; A61B 5/1459; A61B 5/4839; A61B 5/746; A61M 5/1723; A61B 2562/166

(57) ABSTRACT

An integrated physiological monitoring and tissue anchoring device is provided. The device includes a detachable clamp at the distal portion that includes a sensor configured to sense a physiological parameter from the gastrointestinal tract. A handle at the proximal portion of the device is configured to actuate the clamp into a closed configuration in which gastrointestinal tissue is grasped by the clamp. A cable connects the handle to the clamp and has a frangible section near the clamp such that the clamp is detachable from the cable at the frangible section.

### CROSS-REFERENCE TO RELATED APPLICATIONS

The present application claims priority to U.S. Provisional Application No. 63/169,401 filed on Apr. 1, 2021 and incorporated by reference in its entirety herein.

### TECHNICAL FIELD

The present application relates to an integrated physiological monitoring and tissue anchoring device.

### BACKGROUND

One of the challenges in measuring physiological signals for an extended period of time from within the gastrointestinal tract (GI) is anchoring a sensing device to GI tissue. In some applications, measuring physiological signals require coupling to tissue, as in optical sensors such as photoplethysmography (PPG). Various surgical tissue attachment devices exist, such as endoclips, but such devices are purely mechanical tissue attachment devices used for surgical purposes.

### SUMMARY

The present disclosure relates to devices with physiological sensors to measure physiological signals that are integrated with a modular tissue clip. In an aspect, an integrated physiological monitoring and tissue anchoring device is provided. The device has a distal portion and a proximal portion. A clamp is located at the distal portion and comprises at least one arm configured to grasp a side of tissue and an opposing substrate configured to engage an opposing side of the tissue. The substrate comprises a sensor mounted thereon that is configured to sense a physiological parameter from the gastrointestinal tract. A handle is at the proximal portion and is configured to actuate the clamp into a closed configuration in which to the tissue is grasped by the at least one arm such that the sensor contacts the opposing side of the tissue. A cable connects the handle to the clamp and has a frangible section at the distal portion at a location distal to the handle and proximal to the clamp such that the clamp is detachable from the cable at the frangible section.

### BRIEF DESCRIPTION OF DRAWINGS

FIG. 1 is a side view of a device according to an aspect of the present disclosure.

FIG. 2 is a perspective view of the distal portion of a device according to an aspect of the present disclosure.

FIG. 3 is a side view of the distal portion of a device according to an aspect of the present disclosure clipped into tissue.

FIG. 4 is a top view of the distal portion of a device according to an aspect of the present disclosure clipped into tissue.

FIG. 5 is a perspective view of the distal portion of a device according to an aspect of the present disclosure depicting a sensor of the device pressed against tissue while other components of a clamp of the device grasps tissue.

FIG. 6 is a perspective view of a clamp of a device according to an aspect of the present disclosure.

FIG. 7 is a top view of a clamp of a device according to an aspect of the present disclosure.

FIG. 8 a perspective view of clamp of a device according to an aspect of the present disclosure.

FIG. 9 is a side view of a distal portion of a device according to an aspect of the present disclosure.

FIG. 10 is a side view of a distal portion of a device according to an aspect of the present disclosure.

FIG. 11 is a perspective view of a distal portion of a device according to an aspect of the present disclosure.

FIG. 12 is atop view of a clamp of a device according to an aspect of the present disclosure.

FIG. 13 is a side view of the distal portion of the device in communication with a control module and external device according to an aspect of the present disclosure.

### DETAILED DESCRIPTION

As used herein with respect to a described element, the terms “a,” “an,” and “the” include at least one or more of the described elements including combinations thereof unless otherwise indicated. Further, the terms “or” and “and” refer to “and/or” and combinations thereof unless otherwise indicated. By “substantially” is meant that the shape or configuration of the described element need not have the mathematically exact described shape or configuration of the described element but can have a shape or configuration that is recognizable by one skilled in the art as generally or approximately having the described shape or configuration of the described element. A “patient” as described herein includes a mammal, such as a human being. The term “top” and “bottom” refer to the position or orientation of the component as depicted in the drawings. An “ingestible housing” as used herein is a housing that is not just capable of being ingested but rather is understood by one skilled in the art as be suitable for swallowing and entering into the gastrointestinal tract for therapeutic purposes.

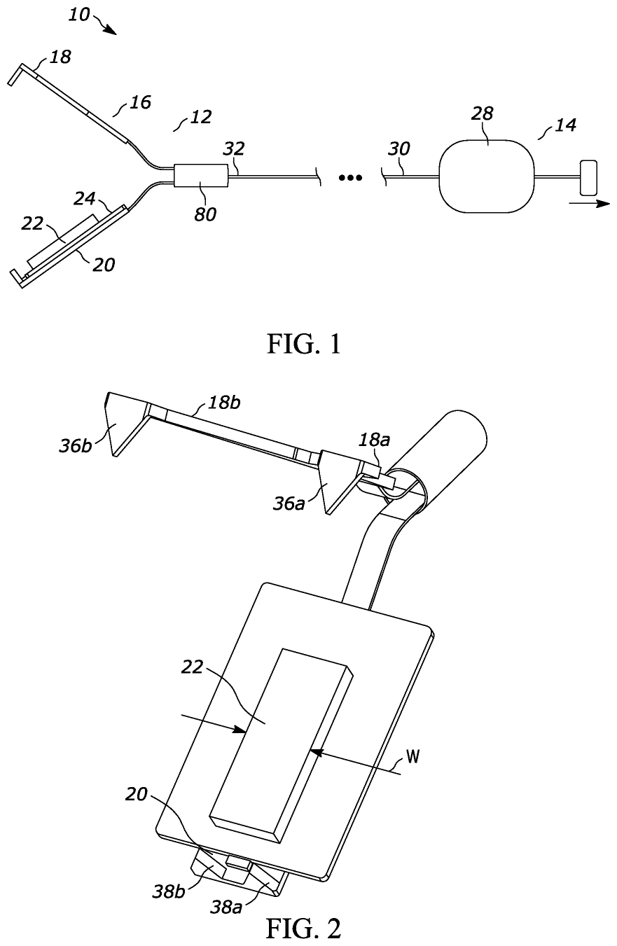

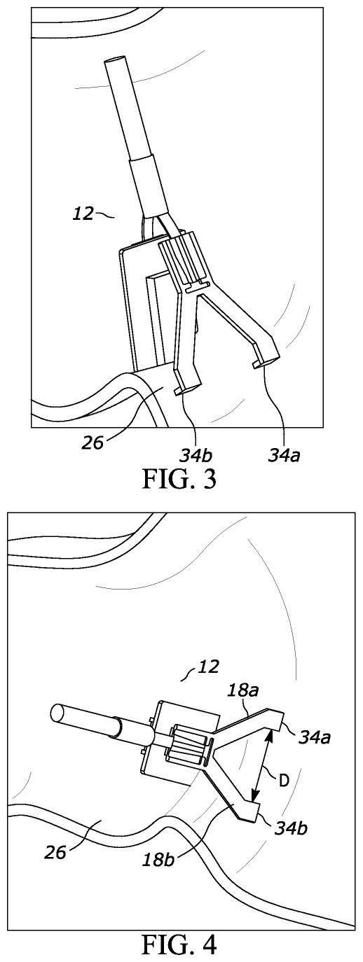

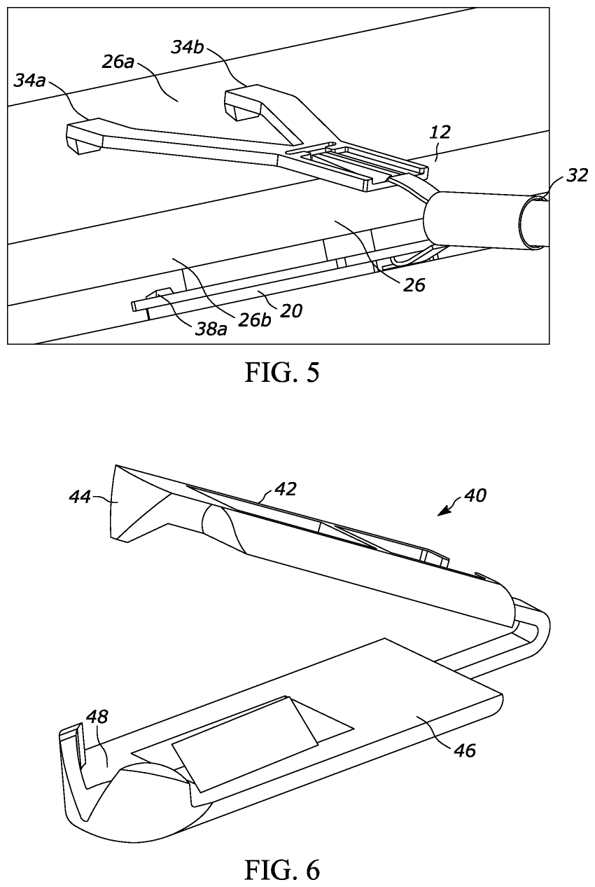

An integrated physiological sensing (also referred to herein as “monitoring”) and tissue anchoring device is provided herein. Referring to FIG. 1-5, in an aspect, a sensing and tissue anchoring device has a distal portion 12 and a proximal portion 14. Distal portion 12 can have a clamp 16 that has at least one arm 18 configured to grasp a side of tissue 26a. Clamp 16 can include opposing substrate 20 configured to engage an opposing side 26b of the tissue. Substrate 20 can have sensor 22 mounted thereon configured to sense a physiological parameter in the GI tract. A printed circuit board 24 can include the electronics to process the sensed physiological parameter. It can also communicate the sensed physiological data to an external device outside the body. Proximal portion 14 can include handle 28 configured to actuate clamp 16 into a closed configuration in which tissue 26 is grasped by the at least one arm 18 such that sensor 22 contacts the opposing side of tissue 26b. Cable 30 can connect handle 28 to clamp 16 and can have a frangible section 32 at distal portion 12 at a location distal to handle 28 and proximal to clamp 16 such that clamp 16 is detachable from cable 30 at frangible section 32.

The device can be inserted through an endoscope into the GI tract (with the cable extending through an endoscope channel, for example). The clamp can be actuated by the handle at the proximal end of the endoscope outside of the patient's body to close the clamp. In particular, the handle can be pulled proximally to close the clamp. Upon closing, the clamp grasps tissue between the at least one arm and the substrate of the clamp. In doing so, the tissue is drawn into contact with the sensor which is important for accurate measurements of physiological parameters in the intestine and other parts of the GI tract. Furthermore, by grasping tissue, the robust nature of the clamp facilitates reliable long-term recordings of physiological parameters (e.g. multiple days). The handle can be pulled further proximally and/or with enough force such that the frangible section of cable snaps, leaving behind the clamp clipped onto tissue. Alternatively, the handle can be pulled proximally with sufficient force to actuate the clamp to grasp tissue and detach at the frangible section of the cable in one motion. The frangible section of the cable near the clamp is an intentionally weaker section that the rest of the cable such that when the handle is pulled proximally with sufficient force, this section fails and breaks the cable free from the clamp.

As stated above, the clamp can have at least one arm configured to grasp tissue. Although FIGS. 2-5 illustrate two arms, the clamp can have any suitable number of arms to grasp tissue. In certain aspects and particularly with respect to an optical sensor, the two arms can be separated by a space as illustrated in FIGS. 2-5 to provide an unobstructed optical path for the optical sensor (e.g. pulse oximeter) into intestinal tissue or other GI tract tissue. For example, the two arms can form a substantially Y-shape as depicted in FIGS. 2-5 and the distance D between the distal end 34a and 34b of arms 18a and 18b can be greater than the width W of sensor 22 to further facilitate an unobstructed optical pathway for an optical sensor. Each of arms 18a and 18b can comprise a respective prong 36a and 36b at respective distal end 34a and 34b configured to engage and grasp tissue 26a. Similarly, substrate can comprise one or more prongs 38 at a distal end thereof configured to engage and grasp the opposing side of tissue 26b.

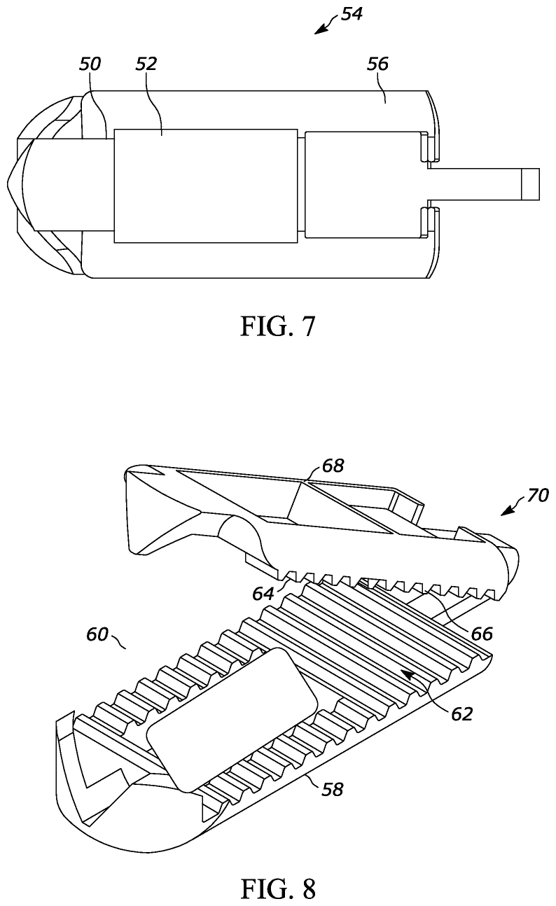

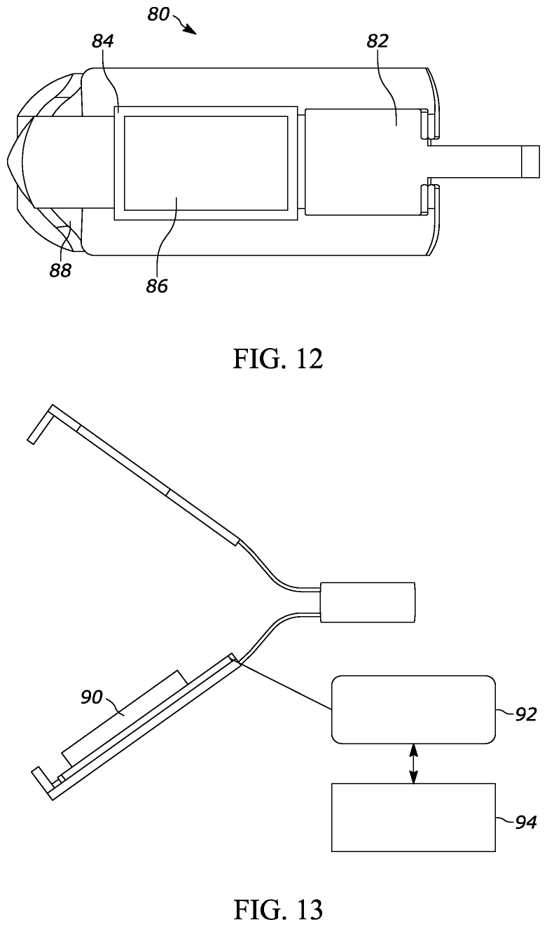

In certain aspects and with respect to FIGS. 6 and 7, a sensing and tissue anchoring device can have a distal prong that is configured to provide extra “bite” to tissue therebetween to provide more resistance to tissue sliding out of the clamp. In particular, clamp 40 can include a single arm 42 having a sharp distal prong 44 and an opposing substrate 46 having a complimentary shaped opening 48. Once the clamp is actuated, distal prong 44 transitions to a position closer to opening 44 with tissue disposed between the opening and the distal prong. In certain aspects and as depicted in FIG. 12, top and bottom faces of arm 82 of clamp 80 can define window 84 to facilitate an optical path into tissue for an optical sensor 86, such as a reflective pulse oximeter, disposed on substrate 88. Referring to FIG. 7, in certain aspects, the top and bottom face of arm 50 of clamp 54 can define no such window such that arm 50 is a solid body 52 to increase the amount of light reflected through the tissue clamped between arm 50 and the substrate 56. Referring to FIG. 8, in certain aspects, top surface 58 of substrate 60 comprises ridges 62 and/or bottom surface 64 of arm 68 comprises ridges 66 to increase the surface area and “bite” force applied to tissue between the arm and the substrate to provide more resistance to tissue sliding out of clamp 70.

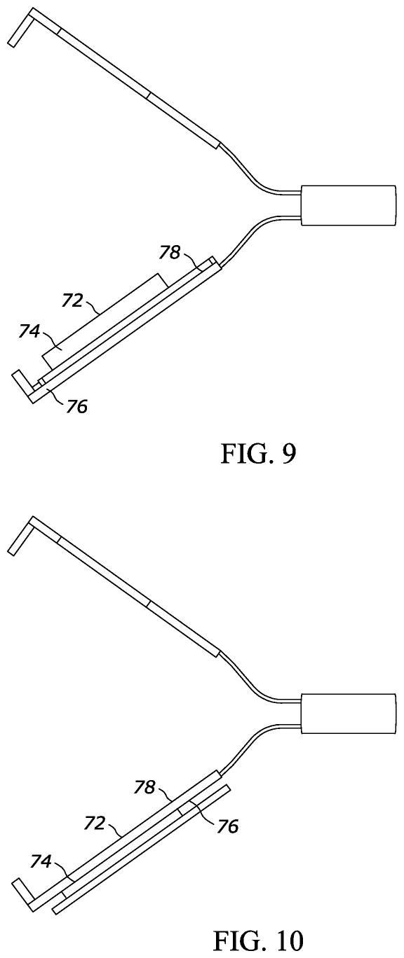

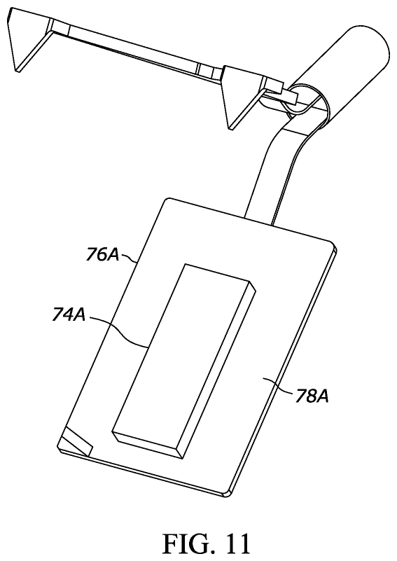

Referring to FIG. 9, top surface 72 of sensor 74 can be proud of top surface 78 of substrate 76. Alternatively, as depicted in FIGS. 10 and 11, sensor 74 can be recessed or flush with top surface 72 of substrate 76. The latter configuration allows other electronic components to be placed on the substrate (or the printed circuit board) outside of the region of the substrate enclosed by the clamp in a closed configuration but ensures contact sufficient contact between the sensor and the tissue grasped by the clamp. The sensor can be a reflective pulse oximeter, an inertial measurement unit such as an accelerometer or a gyroscope, an electrocardiogram (ECG) sensor, a photoplethysograph (PPG) sensor, a temperature sensor, or combinations thereof. Such sensors are exemplary and other sensor(s) can be included in the device that are suitable for measuring physiological parameters from the GI tract including the stomach and/or intestine.

Referring to FIG. 13, in certain aspects, sensor 90 of the device can be in communication with control module 92, that can include, for example, a microprocessor or controller to process sensor data, a battery to power the system, a radio/antenna to wirelessly communicate data with a receiver/external device outside the body, or combinations thereof. Although FIG. 13, only depicts one module, these different components could be part of separate module(s) in communication with other module(s). For example, the rest of the electronics could be on another PCB or multiple PCBs in a module tethered to the clamp. The controller can be operatively coupled to sensor and can be programmed to receive the physiological parameter measured by the sensor that is indicative of a medical condition and prepare the physiological parameter for transmission from the subject, analysis of the physiological parameter, or both.

The controller can be operably coupled to a drug dispenser to actuate release of drug to mitigate the medical condition. For example, the controller can be programmed to actuate release of a medication from the drug dispenser into the patient in response to a determination that the physiological parameter falls outside a threshold value or range for the at least one physiological parameter. The controller can also be programmed to preemptively actuate release of the medication from the drug dispenser into the patient in response to the determination such that the medication is delivered prior to the patient experiencing the medical condition. The sensor can be configured to detect an individual physiological parameter, a combination of physiological parameters, trends in an individual physiological parameter, or trends in a plurality of physiological parameters. The controller can be programmed to actuate release of the medication from the drug dispenser into the patient in response to a determination that the individual physiological parameter, the combination of physiological parameters, trends in the individual physiological parameter, or trends in the plurality of physiological parameters meet a pre-defined condition. The controller can also be programmed to preemptively actuate release of the medication from the drug dispenser into the patient in response to the determination such that the medication is delivered prior to the patient experiencing the medical condition.

For example, the controller can be programmed to send a control signal to a drug delivery dispenser as part of the device or system in response to a measurement of a physiological signal by the sensor that deviate beyond clinically established unsafe levels. One example of this application is a pulse oximeter sensor integrated into the device to measure blood oxygen saturation, heart rate, respiratory rate, or combinations thereof from within the GI tract. In the case of an opioid overdose, the device can detect changes in physiological values (e.g. a decrease in blood oxygen saturation and/or a decrease in respiratory rate) and command a drug delivery dispenser to release an opioid antagonist (e.g. nalmefene or naloxone) to mitigate the effects of the overdose to prevent serious injury or death. In particular, upon the sensor detecting a physiological indication of an opioid overdose, the drug dispenser can release a rescue medication as well as send out alerts to the patient and/or a caregiver. Non-limiting examples of suitable drug dispensers include MEMS drug delivery, valve systems, osmotic plug pistons, electrolytical pumps, or combinations thereof. In certain aspects, the drug dispenser can be a non-refillable reservoir such that it does not include any ports or similar structures that allow the drug dispenser to be re-filled from a location external to the patient's body, such as a syringe containing an opioid antidote that is injected into the drug dispenser from outside the patient's body. The device can be used for other indications and medications and opioid overdose and an opioid antidote is provided as one example.

The device can include a radio that is configured to communicate with external device 94, wherein the controller is configured and programmed to control the operation of the radio to send an alert to external device 94 in response to determining that the subject is experiencing a medical condition. In certain aspects, the controller can be configured and programmed to control the operation of the radio to preemptively send an alert to the external device in response to receiving the physiological parameter such that alert is sent prior to the subject experiencing the medical condition. In addition to the integrated monitoring and anchoring device being delivered and attached onto intestinal or other GI tissue endoscopically, an alternative configuration is a small ingestible housing, such as a capsule or tablet, containing all the components that can be swallowed. An enteric coating of the capsule can dissolve in the intestines and the components can unfold. The clamp can be spring loaded in an open position such that upon deploying in the intestines, the spring (indicated as 80 in FIG. 1) can be released to close the clamp, thereby grasping tissue and anchoring the device to the intestinal wall. A mucoadhesive can be disposed on the surface of the sensor to initiate contact between the sensor and tissue before the spring mechanism is released to grasp the tissue, maintaining the sensor against the tissue.

Each of the disclosed aspects and embodiments of the present disclosure may be considered individually or in combination with other aspects, embodiments, and variations of the disclosure. Further, while certain features of embodiments and aspects of the present disclosure may be shown in only certain figures or otherwise described in the certain parts of the disclosure, such features can be incorporated into other embodiments and aspects shown in other figures or other parts of the disclosure. Along the same lines, certain features of embodiments and aspects of the present disclosure that are shown in certain figures or otherwise described in certain parts of the disclosure can be optional or deleted from such embodiments and aspects. Additionally, when describing a range, all points within that range are included in this disclosure.

What is claimed is:

1. An integrated physiological monitoring and tissue anchoring device having a distal portion and a proximal portion, the device comprising:
a clamp at the distal portion comprising:
at least one arm configured to grasp a side of tissue and having a top face and a bottom face;
an opposing substrate configured to engage an opposing side of the tissue and comprising a sensor mounted thereon that is configured to sense a physiological parameter from the gastrointestinal tract;
a handle at the proximal portion configured to actuate the clamp into a closed configuration in which the tissue is grasped by the at least one arm such that the sensor contacts the opposing side of the tissue; and
a cable connecting the handle to the clamp and having a frangible section at the distal portion at a location distal to the handle and proximal to the clamp such that the clamp is detachable from the cable at the frangible section.

2. The device of claim 1, wherein the at least one arm is two arms separated by a space.

3. The device of claim 2, wherein the two arms form a substantially Y-shape.

4. The device of claim 2, wherein the distance between the two arms is greater than width of the sensor.

5. The device of claim 2, wherein the each of the two arms comprises a prong at the distal end thereof configured to engage and grasp the side of the tissue.

6. The device of claim 1, wherein the substrate comprises a prong at a distal end thereof configured to engage and grasp the opposing side of the tissue.

7. The device of claim 1, wherein the at least one arm comprises a single arm having a prong at a distal end thereof.

8. The device of claim 7, wherein the distal end of the substrate defines an opening having a shape and size complimentary to the prong of the single arm.

9. The device of claim 8, wherein the prong of the single arm and the complimentary opening of the substrate are substantially V-shaped.

10. The device of claim 1, wherein the top surface of the substrate comprises a plurality of ridges.

11. The device of claim 10, wherein the bottom surface of the at least one arm comprises a plurality of ridges.

12. The device of claim 1, wherein the sensor is a reflective pulse oximeter.

13. The device of claim 1, wherein the sensor is mounted on a printed circuit board which, in turn, is mounted on the substrate.

14. The device of claim 1, wherein the top surface of the sensor is substantially flush with the top surface of the substrate.

15. The device of claim 1, wherein the top surface of the sensor is proud of the top surface of the substrate.

16. The device of claim 1, wherein the at least one arm comprises a solid body with no window defined by the top face and the bottom face of the at least one arm.

17. The device of claim 1, wherein the top face and the bottom face of the at least one arm defines a window extending therethrough.

18. The device of claim 1, further comprising a controller operatively coupled to the sensor, the controller programmed to receive the physiological parameter and prepare the physiological parameter for transmission from the subject, analysis of the physiological parameter, or both.

19. The device of claim 19, wherein the device comprises a drug dispenser operably coupled to the controller, the controller programmed to actuate release of a medication from the drug dispenser into the patient in response to a determination that the physiological parameter falls outside a threshold value or range for the at least one physiological parameter or in response to a determination that the physiological parameter meets a pre-defined condition.

20. A system comprising:
the device of claim 1;
a radio configured to communicate with an external device; and
a controller programmed to control the operation of the radio to send an alert to the external device in response to determining that the subject is experiencing a medical condition.
